(19) United States
(12) Patent Application Publication
(10) Pub. No.: US 2012/0232139 A1
(43) Pub. Date: Sep. 13, 2012

(54) COMPOSITION FOR OCULAR TOPICAL ADMINISTRATION TREATMENT OCULAR HYPERTENSION AND GLAUCOMA

(76) Inventors: Ryuji Ueno, Montgomery, MD (US); Junichi Kawasaki, Chiyoda-ku (JP); Tadashi Hayashi, Chiyoda-ku (JP)

(21) Appl. No.: 13/393,657

(22) PCT Filed: Sep. 1, 2010
(86) PCT No.: PCT/JP2010/064919

§ 371 Date: May 14, 2012

Related U.S. Application Data
(60) Provisional application No. 61/238,859, filed on Sep. 1, 2009.

(51) Int. Cl. A61K 31/215; A61P 27/06; A61P 27/02

(52) U.S. Cl. 514/530

(57) ABSTRACT

The present invention provides a composition for ocular topical administration for treating ocular hypertension and glaucoma, comprising latanoprost as an active ingredient, and (a) a polyol and/or sugar alcohol, (b) a nonionic surface active agent, and (c) an edetic acid compound. The composition of the present invention comprises lower amount of preservatives such as benzalkonium chloride comparative to the conventional product and therefore, can reduce incidence of the adverse side effects caused by the preservatives. In addition, the composition of the present invention can be stored stably at room temperatures for a long term.

### FIELD OF THE INVENTION

The present invention relates to a composition comprising latanoprost as an active ingredient for ocular topical administration for treating ocular hypertension and glaucoma.

### ART RELATED

Preservatives for compositions for ocular topical administration are required to have enough antimicrobial activities against bacteria and fungi as well as to be highly safe that do not affect or less affect badly on ocular tissues such as corneal epithelium. Preservatives per se are also required to be stable. In addition, a preservative preferably interacts with the other ingredients in the composition and helps to provide a composition wherein the ingredients are dissolved or dispersed in a vehicle or base. At present, benzalkonium chloride is most popular in the preservatives used in commercially available eye drop products.

Preservatives have been reported to be the main cause of corneal and conjunctival disorders and from the point of view of safety, the concentration of a preservative such as benzalkonium chloride is preferably less than 0.01%. Recently, it has been reported that blood-aqueous barrier breakdown and macular edema, especially, Cystoid Macular Edema (CME) are caused by preservatives in ophthalmic drops (Non-Patent Literatures 1 and 2).

Latanoprost (general name) is the active ingredient of Xalatan®, an ophthalmic solution for the treatment of ocular hypertension and glaucoma available on the market. Xalatan® eye drops comprise benzalkonium chloride as a preservative at a concentration of 0.02% (Non-Patent Literature 3). Side effects such as corneal and conjunctival disorders and CME due to thus high concentration of benzalkonium chloride have been reported.

On the other hand, latanoprost is highly fat soluble and therefore, it is difficult to provide a stable and uniform ophthalmic solution comprising latanoprost by removing or reducing the amount of benzalkonium chloride from Xalatan® eye drops. Some stable and uniform formulations comprising latanoprost have been reported (For example, Patent Literatures 1 and 2).

Further, according to the package insert of Xalatan®, the drug should be protected from light and stored under refrigeration at 2° to 8° C. That is, latanoprost in Xalatan® eye drops is not sufficiently stable at room temperatures and therefore, the product is required to be stored in a cold dark place. The art wanted to improve this point.
Patent Literature 1: WO2004/037267
Patent Literature 2: JP2004-123729A
Non-Patent Literature 1: The 105th annual meeting of the Japanese Ophthalmological Society, Program page 112
Non-Patent Literature 2: OSN Supersite, Top Stories, 97/10/02
Non-Patent Literature 3: Xalatan® package insert.
   Those references as above are incorporated herein by reference.

### SUMMARY OF THE INVENTION

### Problems to Be Solved by the Invention

An object of the present application is to provide a composition for ocular topical administration or an ophthalmic composition for the treatment of ocular hypertension and glaucoma comprising latanoprost as an active ingredient that can be stored stably at room temperatures for a long term and comprises lower amount of preservatives such as benzalkonium chloride comparative to the conventional product.

### Means for Dissolving the Problem

The inventors had found that a stable ophthalmic composition can be prepared by supplementing a polyol and/or a sugar alcohol, a nonionic surface active agent and edetic acid or a salt thereof in an ophthalmic solution comprising latanoprost and that the amount of a ingredient that functions as a preservative and also as a dissolving agent such as benzalkonium chloride in the composition can be reduced from those used in conventional compositions. Thus, the applicant had completed the present invention.

The present provides the followings:
(1) A composition for ocular topical administration for treating ocular hypertension and glaucoma, comprising latanoprost as an active ingredient and further comprising:

(a) a polyol and/or sugar alcohol,

(b) a nonionic surface active agent, and

(c) an edetic acid compound.
(2) The composition of (1), wherein the polyol is glycerine, and sugar alcohol is mannitol.
(3) The composition of (1), wherein the nonionic surface active agent is Polysorbate 80.
(4) The composition of (1), wherein the edetic acid compound is disodium edetate and/or a hydrate thereof.
(5) The composition of (1), further comprising a preservative.
(6) The composition of (1), wherein the preservative is benzalkonium chloride.
(7) The composition of (6), wherein the amount of benzalkonium chloride in the composition is 0.001-0.01 w/v %.
(8) The composition of (7), which is for the treatment of a subject who is suffered from or likely to be suffered from corneal and conjunctival disorders or macular edema in addition to ocular hypertension and glaucoma.
(9) The composition of any one of (1)-(8), which is an ophthalmic solution.
(10) The composition of any one of (1)-(9), which is stored at room temperatures.
(11) An improvement of a composition for ocular topical administration comprising latanoprost as an active ingredient for treating ocular hypertension and glaucoma, which comprises supplementing the composition with:

(a) a polyol and/or a sugar alcohol,

(b) a nonionic surface active agent, and

(c) an edetic acid compound.
(12) A method for storing the composition for ocular topical administration comprising latanoprost, which comprises storing the composition of any one of (1)-(9) at room temperatures.
(13)A method for treating ocular hypertension and glaucoma, which comprises administering the composition of any one of (1)-(10) topically to the eyes of a subject in need of the treatment of ocular hypertension and glaucoma.
(14) Use of a combination of latanoprost as an active ingredient with the followings:

(a) a polyol and/or a sugar alcohol,

(b) a nonionic surface active agent, and

(c) an edetic acid compound

for the manufacture of a composition for treating ocular hypertension and glaucoma for ocular topical administration.

### Effect of the Invention

The composition for ocular topical administration can be stored at room temperatures for a long term. In addition, the amount of the ingredient that acts as a preservative and also as a dissolving agent, such as benzalkonium chloride, in the composition can be greatly reduced comparative to the amount in the conventional latanoprost ophthalmic solution.

### Most Preferred Embodiment

Latanoprost is a prostaglandin analogue. Its chemical name is (+)-isopropyl-(Z)-7[(1R,2R,3R,5S)3,5-dihydroxy-2-[(3R)-3-hydroxy-5-phenylpentyl]cyclopentyl]-5-heptenoate (IUPAC). Latanoprost is a selective FP receptor agonist that is believed to reduce the intraocular pressure (IOP) by increasing the outflow of aqueous humor and therefore, is useful for the treatment of ocular hypertension and glaucoma.

The term “treatment” or “treating” used herein refers to any means of control of a condition including prevention, cure, relief of the condition, attenuation of the condition and arrest of progression.

The ophthalmic composition of the present invention includes any dosage form for topical ocular administration used in the field of ophthalmology. The ophthalmic composition may be in the form of liquid, such as solution, emulsion and dispersion, semi-liquid such as gel and eye ointment. Eye drops that may be solution, emulsion or dispersion may preferably be used. The ophthalmic composition can be prepared in accordance with conventional means known in the relevant technical field. Eye drops that may be emulsion, dispersion or solution are preferably used. The concentration of latanoprost in the ophthalmic composition of the present invention may be in general about 0.001-0.01 w/v %, and may preferably be similar to that of the commercially available Xalatan® ophthalmic solution, i.e. about 0.005 w/v %. The ophthalmic composition of the present invention comprising latanoprost may be administered to an eye of the subject one to four times per day, preferably one to three times per day, and more preferably, one or two times per day.

The polyol used in this invention is a polyvalent alcohol and bi- and tri-valent alcohols are preferable. Examples of polyols may include glycerin, polyethylene glycol and propylene glycol and glycerin is especially preferable. The amount of the polyol in the ophthalmic composition of the present invention may be about 0.1-10 w/v % in general and preferably, about 0.5-5 w/v %.

The sugar alcohols used in the present invention is an alcohol obtained by hydrogen reduction of the aldehyde group of a saccharide molecule. Examples may comprise sorbitol, mannitol, maltitol, lactitol, palatinit, xylitol and erythritol; and sugar alcohol solution derived from corn starch, i.e. a mixture of sorbitol, sorbitan, mannitol and hydrogenated starch hydrolysate, hydrogenated maltose starch syrup, i.e. a mixture of maltitol, sorbitol and oligosaccharide alcohol. Mannitol is especially preferable. The amount of the sugar alcohol added to the pharmaceutical composition of the present invention may generally be about 0.1-10 w/v % and preferably, about 0.5-5 w/v %.

In order to satisfy the object of the present invention, the composition must comprise a polyol such as glycerine as ingredient (a). In combination with the polyol, a sugar alcohol such as mannitol may preferably be supplemented in the composition. The combination of glycerine and mannitol is especially preferable.

Non-ionic surface active agent represents a surface active agent having no group that is easily ionized. Examples of preferred nonionic surface active agents may include polyoxyethylene sorbitan fatty acid esters such as polysorbate 20, 60 and 80; polyoxyethylene castor oil derivatives such as polyoxyethylene castor oil 35, polyoxyethylene hydrogenated castor oil 40 and polyoxyethylene hydrogenated castor oil 60; polyoxyethylene alkylethers; polyoxyethylene polyoxypropyleneglycols; and polyoxyl stearates. Polysorbate 80, i.e. polyoxyethylene sorbitan monooleate is especially preferable. The amount of the nonionic surface active agent in the ophthalmic composition of the present invention may be about 0.01-1 w/v % in general and preferably, about 0.05-0.5 w/v %.

“Edetic acid compound” in this invention represents a compound selected from edetic acid (ethylene diamine tetra-acetic acid), a salt thereof or a chilate of the acid and 1-4 valent metal ion, and a hydorate thereof. Examples of edetic acid compounds may include edetic acid, monosodium edetate, disodium edetate, trisodium edetate, tetrasodium edetate, calcium disodium edetate, dipoptassium edetate, disodium edetate dihydrate, tetrasodium edetate dihydrate, tetrasodium edetate tetrahydrate. Disodium edetate and its hydrates are preferably used. The amount of the edetic acid compound in the ophthalmic composition of the present invention may be about 0.001-1 w/v % in general and preferably, about 0.01-0.5 w/v %.

The ophthalmic composition of the present invention can be manufactured easily. The latanoprost contained in the composition may stably maintained for long term in the ophthalmic composition even if it is stored at room temperatures. The ophthalmic composition of the present invention may be formulated as a sterile unit dose type product, or a product for single use only.

The ophthalmic composition of the present invention may comprise a preservative. Examples of preservatives may comprise chlorhexidines such as benzalkonium chloride, benzethonium chloride and gluconate chlorohexidine, paraoxybenzoic acid esters such as ethyl paraoxybenzoates and methyl paraoxybenzoates, sorbic acid or its salt such as sorbic acid, potassium sorbate. Benzalkonium chloride is especially preferable. According to the present invention, the amount of the preservative in the ophthalmic composition can be greatly reduced from those contained in conventional products while keeping the stability of the active ingredient at room temperatures for long term.

Xalatan® ophthalmic solution available on the market comprises benzalkonium chloride in an amount of 0.02 w/v %. In contrast, according to the present invention, an ophthalmic composition comprising 0.001 w/v %-0.01 w/v %, more preferably, 0.001 w/v %-0.005 w/v % of benzalkonium chloride can be prepared.

The ophthalmic composition of the present invention may further comprise an additive other than the above described preservatives. The additive may be any of those have been employed in the field of ophthalmology. In the preparation of the eye ointment, the composition may contain, in addition to the above additives, a commonly used eye ointment base.

The ophthalmic composition of the present invention can be stored at room temperatures, for example at temperatures between 15-25° C. The composition of the present invention can keep the active ingredient for long term, as long as about 6 months, preferably 12 months even if it is stored at room temperatures. It can be guaranteed that the ophthalmic composition of the present invention can be stored at room temperatures with maintaining the active ingredient in the composition stably for at least about 3 months.

According to the present invention, latanoprost ophthalmic composition comprising substantially reduced amount of benzalkonium chloride comparative to the amount in the conventional commercially available product, and the composition of this invention can be stored stably at room temperatures. Accordingly, the ophthalmic composition of the present invention shows significantly reduced incidence of side effects such as corneal and conjunctival disorders and CME compared to that induced by Xalatan®, a commercially available latanoprost ophthalmic solution. Accordingly, the ophthalmic composition of the present invention can treat ocular hypertension and glaucoma in a patient suffered from corneal or conjunctival disorders and/or CME more effectively.

The ophthalmic composition of the present invention may further comprise a pharmaceutically active ingredient other than latanoprost in so far as it does not act adverse to the purpose of the present invention. Examples of the pharmaceutically active ingredients may include parasympathomimetic agents such as pilocarpine and carbachol; cholinesterase inhibitors such as physostigmine salicylate, distigmine bromide and echothiopate iodide; sympathomimetic agents such as epinephrine, dipivalylepinephrine, clonidine, p-aminoclonidine and brimonidine; β-adrenergic blockers such as betaxolol, levobunolol, timolol and carteolol; prostones such as isopropyl unoprostone, prostaglandin compounds such as travoprost, bimatoprost and tafluprost; tropicamide and the like. Among these pharmaceutically active agents, timolol is especially preferable. In the preparation that contains two or more active ingredients, the amount of each ingredient may be determined appropriately according to the therapeutic effects and safety of each ingredient.

The present invention will be described in more detail with reference to the following examples, which is not intended to limit the scope of the present invention.

EXAMPLE 1

Test solution 1 was obtained by dissolving the ingredients in an amount shown below (w/v %) in purified water.

| | |
|---|---|
| 0.005% | latanoprost |
| 0.2% | polysorbate 80 |
| 2.5% | concentrated glycerine |
| 0.05% | disodium edetate dihydrate (sodium edetate |
| | (Japanese Pharmacopoeia)) |
| 0.002% | benzalkonium chloride |

Thus obtained test solution 1 was filled in a low density polyethylene (LDPE) container and stored for two weeks at 55° C. The concentration of latanoprost in the test solution at the starting of the storage and after two weeks storage were determined by means of liquid chromatography.

The concentration was measured by means of liquid chromatography in the manner as follows:

Exactly 1 ml of the sample was precisely measured and exactly 1 ml of an internal standard solution was added to the sample to give a sample measurement solution. Separately, standard latanoprost was weighted precisely around 0.0125 g, added with acetonitrile (liquid chromatograph grade) to give solution of precise total amount of 50 ml. One milliliter (1 ml) of thus obtained solution was added with 5 ml of the internal standard solution and 10 ml of distilled water (liquid chromatograph grade) to give a standard solution. Each 10 μl of the sample measurement and standard solution was loaded on the liquid chromatograph and determined the content of the compound by internal standard method.

### HPLC Measurement Condition

Detector: fluorescent spectrometer (measurement wavelength: 210 nm)
Column: A stainless tube having about 6 mm of internal diameter and about 15 cm of length, packed with 5 μm octadecylsilyl silica gel for liquid chromatograph
Column temperature: 40° C.
Mobile phase: Mixed solution of acetonitrile(liquid chromatograph grade)/distilled water (liquid chromatograph grade)

### EXAMPLE 2

Test solution 2 consisting of the ingredients as follows was prepared in the same manner as Example 1 except for the amount of concentrated glycerine was changed to 1.9 w/v %, and 1 w/v % of mannitol was added.

| | |
|---|---|
| 0.005% | latanoprost |
| 0.2% | polysorbate 80 |
| 1.9% | concentrated glycerine |
| 1.0% | mannitol |
| 0.05% | disodium edetate dihydrate (sodium edetate, |
| | Japanese Pharmacopenia)) |
| 0.002% | benzalkonium chloride |

Thus obtained test solution 2 was stored in the same manner as Example 1 at 55° C. and the concentration of latanoprost after the storage was measured.

### EXAMPLE 3

Test solution 3 consisting of the ingredients as follows was prepared in the same manner as Example 2 except for the amount of polysorbate 80 was changed to 0.3 w/v %.

| | |
|---|---|
| 0.005% | latanoprost |
| 0.3% | polysorbate 80 |
| 1.9% | concentrated glycerine |
| 1.0% | mannitol |
| 0.05% | disodium edetate dihydrate (sodium edetate, |
| | Japanese Pharmacopenia)) |
| 0.002% | benzalkonium chloride |

Thus obtained test solution 3 was stored in the same manner as Example 1 at 55° C. and the concentration of latanoprost after the storage was measured.

### EXAMPLE 4

Test solution 4 consisting of the ingredients as follows was prepared in the same manner as Example 2 except for the amount of disodium edetate was changed to 0.1 w/v %.

| | |
|---|---|
| 0.005% | latanoprost |
| 0.2% | polysorbate 80 |
| 1.9% | concentrated glycerine |
| 1.0% | mannitol |
| 0.1% | disodium edetate dihydrate (sodium edetate, |
| | Japanese Pharmacopenia)) |
| 0.002% | benzalkonium chloride |

Thus obtained test solution 4 was stored in the same manner as Example 1 at 55° C. and the concentration of latanoprost after the storage was measured.

### COMPARATIVE EXAMPLE 1

Xalatan® ophthalmic solution (latanoprost:
0.005%) was used as test solution 5.
0.005% latanoprost
0.02% benzalkonium chloride
   The other ingredients are as follows: disodium hydrogenphosphate, sodium dihydrogen phosphate and sodium chloride(Xalatan® marketed in US package insert).

Test solution 5 was stored in the same manner as Example 1 at 55° C. and the concentration of latanoprost after the storage was measured.

Results are shown in Table 1 below.

TABLE 1
Latanoprost concentration in the solutions stored at 55° C.

amount of
latanoprost vs.
starting amount (%)

| | start | after 2 weeks |
|---|---|---|
| test solution 1 | 100 | 99.6 |
| test solution 2 | 100 | 99.1 |
| test solution 3 | 100 | 99.9 |
| test solution 4 | 100 | 100.5 |
| test solution 5 | 100 | 90.7 |

According to the results as above, the stability of latanoprost was significantly increased by supplementing glycerine and/or mannitol, polysorbate 80 and disodium edetate dihydrate.

1. A composition for ocular topical administration for treating ocular hypertension and glaucoma, comprising latanoprost as an active ingredient and further comprising:
(a) polyol and/or sugar alcohol,
(b) nonionic surface active agent, and
(c) an edetic acid compound.

2. The composition of claim 1, wherein the polyol is glycerine, and sugar alcohol is mannitol.

3. The composition of claim 1, wherein the nonionic surface active agent is Polysorbate 80.

4. The composition of claim 1, wherein the edetic acid compound is disodium edetate and/or a hydrate thereof.

5. The composition of claim 1, further comprising a preservative.

6. The composition of claim 1, wherein the preservative is benzalkonium chloride.

7. The composition of claim 6, wherein the amount of benzalkonium chloride in the composition is 0.001-0.01 w/v %.

8. The composition of claim 7, which is for the treatment of a subject who is suffered from or likely to be suffered from corneal and conjunctival disorders or macular edema in addition to ocular hypertension and glaucoma.

9. The composition of claim 1, which is an ophthalmic solution.

10. The composition of claim 1, which is stored at room temperatures.

11. An improvement of a composition for ocular topical administration comprising latanoprost as an active ingredient for treating ocular hypertension and glaucoma, which comprises supplementing the composition with:
(a) a polyol and/or a sugar alcohol,
(b) nonionic surface active agent, and
(c) an edetic acid compound.

12. A method for storing the composition for ocular topical administration comprising latanoprost, which comprises storing the composition of any one of claims 1-10 at room temperatures.

13. A method for treating ocular hypertension and glaucoma, which comprises administering the composition of claim 1 topically to the eyes of a subject in need of the treatment of ocular hypertension and glaucoma.

14. (canceled)
